(19) United States
(12) Patent Application Publication
(10) Pub. No.: US 2007/0259366 A1
(43) Pub. Date: Nov. 8, 2007

(54) DIRECT PRINTING OF PATTERNED HYDROPHOBIC WELLS

(76) Inventors: Greg Lawrence, Indianapolis, IN (US); Ignacio Sanchez, Plymouth, IN (US); Fred Regnier, West Lafayette, IN (US); Ray DeGrella, Anaheim, CA (US)

Correspondence Address: BOSE MCKINNEY & EVANS LLP;JAMES COLES, 135 N PENNSYLVANIA ST, SUITE 2700, INDIANAPOLIS, IN, 46204, US

(21) Appl. No.: 11/743,913

(22) Filed: May 3, 2007

Related U.S. Application Data
(60) Provisional application No. 60/797,347, filed on May 3, 2006.

(51) Int. Cl. C12Q 1/68; G01N 33/53; C12M 3/00

(52) U.S. Cl. 435/6; 435/7.1; 435/287.2

(57) ABSTRACT

The present invention comprises a diagnostic platform. The platform has a substrate having plurality of hydrophobic wells formed thereon. The hydrophobic wells are directly printed onto the substrate in a test pattern and are adapted to contain a biological sample for conducting a diagnostic assay to identify analytes within the biological sample. The test pattern is divided into equally sized quadrants, each containing an equal number of hydrophobic wells. The hydrophobic wells may be in a single or double well arrangement, as well as may be connected by at least one microfluidic channel.

### CROSS-REFERENCE TO RELATED APPLICATIONS

This application claims the benefit of U.S. Provisional Patent Application Ser. No. 60/797,347, filed May 3, 2006, which is incorporated in its entirety herein by this reference. This application is also related to U.S. patent application Ser. No. 10/726,772, entitled “Adaptive Interferometric Multi-Analyte High-Speed Biosensor,” filed Dec. 3, 2003 (published on Aug. 26, 2004 as U.S. Pat. Publication No. 2004/0166593), which is a continuation-in-part of U.S. Pat. No. 6,685,885, filed Dec. 17, 2001 and issued Feb. 3, 2004, the disclosures of which are all incorporated herein by this reference. This application is further related to U.S. patent application Ser. No. 11/345,462 entitled “Method and Apparatus for Phase Contrast Quadrature Interferometric Detection of an Immunoassay,” filed Feb. 1, 2006; and also U.S. patent application Ser. No. 11/345,477 entitled “Multiplexed Biological Analyzer Planar Array Apparatus and Methods,” filed Feb. 1, 2006; and also U.S. patent application Ser. No. 11/345,564, entitled “Laser Scanning Interferometric Surface Metrology,” filed Feb. 1, 2006; and also U.S. patent application Ser. No. 11/345,566, entitled “Differentially Encoded Biological Analyzer Planar Array Apparatus and Methods,” filed Feb. 1, 2006, the disclosures of which are all incorporated herein by this reference.

### TECHNICAL FIELD

The present invention generally relates to proteomics and diagnostic devices, and more particularly to the direct printing of patterned wells and/or microfluidic channels on detectable surfaces.

### BACKGROUND OF THE INVENTION

In many chemical, biological, medical, and diagnostic applications, it is desirable to detect the presence of specific molecular structures in a sample. Many molecular structures such as cells, viruses, bacteria, toxins, peptides, DNA fragments, and antibodies are recognized by particular receptors. Biochemical technologies including gene chips, immunological chips, and DNA arrays for detecting gene expression patterns in cancer cells, exploit the interaction between these molecular structures and the receptors. [For examples see the descriptions in the following articles: Sanders, G. H. W. and A. Manz, Chip-based Microsystems for genomic and proteomic analysis. Trends in Anal. Chem., 2000, Vol. 19(6), p. 364-378. Wang, J., From DNA biosensors to gene chips. Nucl. Acids Res., 2000, Vol. 28(16), p. 3011-3016; Hagman, M., Doing immunology on a chip. Science, 2000, Vol. 290, p. 82-83; Marx, J., DNA Arrays reveal cancer in its many forms. Science, 2000, Vol. 289, p. 1670-[672]. These technologies generally employ a stationary chip prepared to include the desired receptors (those that interact with the target analyte or molecular structure under test). Since the receptor areas can be quite small, chips may be produced which test for a plurality of analytes. Ideally, many thousand binding receptors are used to provide a complete assay. When the receptors are exposed to a biological sample, only a few may bind a specific protein or pathogen. Ideally, these receptor sites are identified in as short a time as possible.

Scientists and clinicians often use biological molecules as tools of molecular or clinical diagnostics, high throughput screening, drug discovery, cellular function, biological mechanisms, genetic manipulation or screening as well as numerous other applications. In many instances, biomolecules are employed, ranging in mass from less than one kilo-Daltons to several million Daltons. At the core of these tools is the detection or monitoring of molecular binding events.

Assays developed through the detection of biomolecular interactions or more broadly macromolecular interactions, have been useful tools for scientists and clinicians, particularly due to their sensitivity, their ability to inexpensively achieve fast results, as well as their potential for screening many molecular systems simultaneously. As such, the usefulness of a given assay system is typically a function of many different factors including, but not limited to, cost, speed, throughput, accuracy, precision, sensitivity, testing range, reliability, parallelism, ease of use and flexibility. Likewise, the appropriateness of a given assay system or tool is highly dependent on the specific needs of the researcher or clinician.

One such technology for screening a plurality of molecular structures is the so-called immunological compact disc, which, in the immunological application, simply includes an antibody microarray. [For examples, see the descriptions in the following articles: Ekins, R., F. Chu, and E. Biggart, Development of microspot multi-analyte ratiometric immunoassay using dual flourescent-labelled antibodies. Anal. Chim. Acta, 1989, Vol. 227, p. 73-96; Ekins, R. and F. W. Chu, Multianalyte microspot immunoassay—Microanalytical “compact Disc” of the future. Clin. Chem., 1991, Vol. 37(11), p. 1955-1967; Ekins, R., Ligand assays: from electrophoresis to miniaturized microarrays. Clin. Chem., 1998, Vol. 44(9), p. 2015-2030]. Conventional fluorescence detection is employed to sense the presence in the microarray of the molecular structures under test. These processes; however, are often characterized by the known deficiencies of fluorescence detection and fail to provide rapid repetitive scanning techniques.

Other approaches to immunological assays employ traditional Mach-Zender interferometers that include waveguides and grating couplers. [For examples, see the descriptions in the following articles: Gao, H., et al., Immunosensing with photo-immobilized immunoreagents on planar optical wave guides. Biosensors and Bioelectronics, 1995, Vol. 10, p. 317-328; Maisenholder, B., et al., A GaAs/AlGaAs-based refractometer platform for integrated optical sensing applications. Sensors and Actuators B, 1997, Vol. 38-39, p. 324-329; Kunz, R. E., Miniature integrated optical modules for chemical and biochemical sensing. Sensors and Actuators B, 1997, Vol. 38-39, p. 13-28; Dübendorfer, J. and R. E. Kunz, Reference pads for miniature integrated optical sensors. Sensors and Actuators B, 1997 Vol. 38-39, p. 116-121; Brecht, A. and G. Gauglitz, recent developments in optical transducers for chemical or biochemical applications. Sensors and Actuators B, 1997, Vol. 38-39, p. 1-7]. Interferometric optical biosensors have the intrinsic advantage of interferometric sensitivity, but are often characterized by large surface areas per element, long interaction lengths, or complicated resonance structures. They also can be susceptible to phase drift from thermal and mechanical effects.

One biological compact disc system is disclosed in U.S. application Ser. No. 10/726,772, entitled “Adaptive interferometric multi-analyte high-speed biosensor” and filed Dec. 3, 2003. This application teaches a compact disc platform that contains biological samples that are scanned by a laser head to detect the optical signatures (such as changes in refraction, surface shape, scattering or absorption) of the biological structures bound to its receptors. Typically, the scanning is performed at or near a condition of phase quadrature to maximize sensitivity of the system. The biological compact disc was introduced as a sensitive spinning-disc interferometer that is capable of being operated at a high rate of speed and in a self-referencing manner [see M. M. Varma, H. D. Inerowicz, F. E. Regnier, and D. D. Nolte, “High-speed label-free detection by spinning-disc micro-interferometry,”Biosensors & Bioelectronics, vol. 19, pp. 1371-1376, 2004]. This type of optical biosensor is also capable of generating images of optical parameters, such as fluorescence or reflectance.

Another platform for screening molecular structures is the Gyrolab Bioaffy™ CD microlaboratory, which involves the fabrication of microstructures onto the surfaces of compact discs. The individual fabricated microstructures each have an individual sample inlet and a volume definition chamber that leads to an overflow channel. Moreover, the defined volumes of samples and reagents are applied to a pre-packed column, and the samples and reagents are added to a specific microstructure via an individual inlet or to a group of microstructures via a common channel. Capillary action draws the samples and reagents into the microstructures, where volumes are precisely defined. Additional information on these structures can be found at www.gyros.com, as well as in the publications: Honda et al., Simultaneous Multiple Immunoassays in a Compact Disc-Shaped Microfluidic Device Based on Centrifugal Force, Clinical Chemistry, 51:1955-1961, 2005; and Inganas et al., Integrated Microfluidic Compact Disc Device with Potential Use in Both Centralized and Point-of-Care Laboratory Settings, Clinical Chemistry, 51:1985-1987, 2005.

Many platforms designed to screen for molecular structures require lengthy processing steps that involve disc fabrication techniques, as well as photolithographic methods. As is known within the art, photolithographic methods are typically performed in a series of sophisticated steps that may involve photomask fabrication techniques and/or photoresist patterning procedures (e.g., photoresist spinning, alignment and exposure). In addition, many processes also require sophisticated molding and sealing fabrication processes, thereby adding to their processing times.

There is a need to produce assays or tools based on the detection of biomolecular or macromolecular interactions, which are faster, less expensive, as well as more sensitive and accurate. The purpose of this invention is intended to improve upon and/or to overcome one or more of the shortcomings of the prior art discussed above.

### SUMMARY OF THE INVENTION

In general, the present invention involves the direct printing of hydrophobic materials on silicon or glass surfaces to form patterned wells and microfluidic channels.

According to one general aspect of the present invention, a process for directly printing Microfluidic Channels onto substrates is provided. These Microfluidic Channels exhibit unique physical and/or chemical attributes as compared to previously described hydrophobic materials.

Another aspect of the present invention involves the modification of printed hydrophobic features to facilitate sample flow control, and particularly to identify that the channel's chemical nature is modified in a non-printing second treatment step. According to this embodiment, the surface of the channel is chemically modified to a more hydrophilic surface as a means to control sample flow within the microfluidic channel.

According to one aspect of the present invention, a sample platform having a first surface with a plurality of patterned wells resulting from direct printing of hydrophobic materials on the first surface is provided. According to this aspect of the invention, the platform may be, for example, a silicon or glass wafer with a diameter of about 100 mm.

According to another aspect of the present invention, a diagnostic platform is provided. The platform has a substrate having plurality of hydrophobic wells formed thereon. The hydrophobic wells are directly printed onto the substrate in a test pattern and are adapted to contain a biological sample for conducting a diagnostic assay to identify analytes within the biological sample. The test pattern is divided into equally sized quadrants, each containing an equal number of hydrophobic wells. The hydrophobic wells may be in a single or double well arrangement, as well as may be connected by at least one microfluidic channel.

In yet another aspect of the present invention, a diagnostic platform for identifying analytes in a biological sample is provided. The platform comprises a substrate having a test pattern of between about 10 and about 10,000 hydrophobic wells and a microfluidic channel adapted to connect a pair of hydrophobic wells within the test pattern. The test pattern is directly printed onto the substrate, and the microfluidic channel is adapted to facilitate the transport of the biological sample to the hydrophobic wells to enable a biomolecular binding interaction.

In still another aspect of the present invention, a process for preparing a diagnostic platform for identifying analytes in a biological sample is provided. The process comprises directly printing a test pattern of hydrophobic wells onto a substrate, imaging protein spots into the hydrophobic wells and delivering the sample to the protein spots via at least one microfluidic channel coupled to a pair of hydrophobic wells printed on the substrate. The protein spots are configured to conduct a diagnostic assay on the biological sample within the wells to identify analytes contained therein, and the test pattern is printed onto the substrate by a one-step printing technique selected from at least one of pad printing and screen printing.

### BRIEF DESCRIPTION OF DRAWINGS

The above-mentioned aspects of the present teachings and the manner of obtaining them will become more apparent and the teachings will be better understood by reference to the following description of the embodiments taken in conjunction with the accompanying drawings, wherein:

FIG. 1 is a top view of an exemplary detection platform having a test pattern of 84 hydrophobic wells in accordance with the present teachings;

FIG. 2 is a top view of an exemplary detection platform having a test pattern of 260 hydrophobic wells in accordance with the present teachings;

FIG. 3 is a top view of an exemplary detection platform having a test pattern of 108 hydrophobic double wells in accordance with the present teachings; and

FIG. 4 is a top view of an exemplary detection platform having a test pattern of 96 hydrophobic double wells in accordance with the present teachings.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

In one embodiment of the present invention, there is disclosed a direct printing method to form various patterns with defined wells and microfluidic channels. In certain aspects of the present invention, the wells are physically separated from one another, while in other aspects the wells are physically connected or adjoined to one another by microfluidic channels. Moreover, some embodiments of the present invention provide a platform that enables the simultaneous analysis of multiplexed arrays.

The present invention is also directed to a device and method for facilitating the transport of analytes in a fluid, and particularly a fluid used with a biological compact disc platform. According to these exemplary embodiments, the apparatus and method(s) facilitate the transport of biological molecules to enable a biomolecular binding interaction including, but not limited to, antibody-antigen, DNA and/or RNA hybridization, DNA and/or RNA interactions with proteins, cell surface constituents, enzymatic and substrate interactions, or other known macromolecule interactions. Specific applications of the present invention include, but are not limited to, diagnostic testing, drug development, microarray technology, high throughput screening, or the development of other research tools known to those skilled in the art.

Typically, biological molecule interactions are exploited to gain insight into a given sample composition. One specific example, for instance, includes immunochemical binding events that are often the basis of clinical diagnostic testing. According to this exemplary illustration, an antibody specifically binds to an antigen detectable by numerous methods. These molecules tend to be large species that have limited diffusion rates. As such, biomolecular binding events with these molecules are limited by the time required for the binding molecule to diffuse to the binding site. Therefore, the usefulness of the binding interaction as a scientific or diagnostic tool is often limited by the mass-transfer if facilitated only by diffusion. Using immunoassay interactions as an example, for a given amount of time, the rate of diffusion limits the amount of antigen that can be bound to antibody. This limits the sensitivity of the tool as a diagnostic or research test. As increasingly sensitive methods are developed to recognize molecular binding events, particularly in instances in which large molecules are employed, mass-transfer limitations due to diffusion become determining in terms of overall sensitivity. Examples include the interferometric detection methods of U.S. application Ser. No. 10/726,772 filed Dec. 3, 2003 and published on Aug. 26, 2004 as U.S. Publication No. 2004/0166593 entitled “Adaptive Interferometric Multi-Analyte High-Speed Biosensor”; U.S. Patent Application No. 60/774,273 filed Feb. 16, 2006 entitled “In-Line Quadrature Interferometric Detection”; U.S. patent application Ser. No. 11/345,462 entitled “Method and Apparatus for Phase Contrast Quadrature Interferometric Detection of an Immunoassay,” filed Feb. 1, 2006; and also U.S. patent application Ser. No. 11/345,477 entitled “Multiplexed Biological Analyzer Planar Array Apparatus and Methods,” filed Feb. 1, 2006; and also U.S. patent application Ser. No. 11/345,564, entitled “Laser Scanning Interferometric Surface Metrology,” filed Feb. 1, 2006; and also U.S. patent application Ser. No. 11/345,566, entitled “Differentially Encoded Biological Analyzer Planar Array Apparatus and Methods;” all of which are incorporated herein by reference.

In certain embodiments, assay systems can permit investigation of multiple analytes simultaneously and/or in parallel. Ideally, embodiments having parallel processing should be transparent and convenient for the user of the assay system. Thus, in some applications it is useful to segregate fractions of the applied sample to distinct substrate addresses in which a specific biomolecular interaction can be investigated. Additionally, the assays can also exploit the use of separate control sites to compare measured binding events. For analytical and clinical chemistry methods, the use of a control has numerous implications including, but not limited to, test validity, accuracy, and precision. The use of a control also provides a method for adjusting to non-specific responses. In these and other instances, a means to control the sample flow in a precise and defined pathway is advantageous.

For large molecules, such as various biological molecules, diffusion to a binding site is limited by diffusion distance in a static assay. To compensate, static incubations in which low concentrations of analyte are to be detected require more incubation time. Greater incubation time, however, negatively impacts speed and sample throughput. In this context, static incubation also limits the dynamic range of the assay. Dynamic range is important if there is a need to differentiate between various concentrations of antigen contained within a sample. For samples that contain a high level of antigen, the binding sites on the substrate are quickly saturated with the antibody-antigen complex. With fixed and limited incubation times, the binding event is not rate limiting, and the amount of immobilized antibody is constant; therefore, saturation of the antibody-antigen complex is a function of antigen diffusion rate. Once the substrate becomes saturated, however, samples that contain higher levels of antigen result would not be differentiated by the assay system (leveling of a dose dependent curve). On the other hand, at lower concentrations of antigen, diffusion to the antibody becomes the limiting physical event. In this instance, the dynamic range of the assay is truncated due to the assay system's inability to distinguish results that are below the antigen diffusion rate. Therefore, the sensitivity and dynamic range of the assay system is limited by a mass transfer event relating to the physical ability of the antigen to interact with the immobilized antibody, and not instrument sensitivity. With other assay systems, instrument sensitivity or detectable limits define assay sensitivity as well as the dynamic range of the system. In the instance of spinning disc interferometry methods (as disclosed in the previously incorporated by reference patent applications), diffusion limited mass transfer becomes limiting unless another mechanism for mass transport is considered. Moreover, in practice, the rate of diffusion of a species is inversely related to the size of the species. For diffusion limited binding events, increasing the volume that contains the antigen does not increase the amount of binding observed unless the incubation time is also increased.

It should be understood and appreciated herein that various embodiments of the present invention are capable of providing a practical solution to many problems commonly experienced within the diagnostic sampling industry. More particularly, rather than processing one sample at a time, the present invention enables a large number of samples to be processed simultaneously on a single disc. Moreover, the connection of double wells by open microfluidic channels provides a useful way to simultaneously analyze arrays of materials having highly different levels of sensitivity. As such, different analyses can be performed on the same disc either between the wells or within the same well. Finally, the present invention also allows the microarrays to be printed inside the wells and/or within the microfluidic channels, which thereby results in the formation of highly multiplexed and high-throughput arrays. In addition, the present invention can be implemented using a one-step printing procedure, which simplifies the manufacturing process and significantly reduces processing time. Through the design and implementation of the various embodiments of the herein disclosed apparatus, the process of fluid dynamics facilitates transport and subsequent binding and detection of low concentrations of single or multiple analytes contained within a biological sample. It should be understood that it is contemplated as within the scope of the present invention that any given embodiment might provide one or more of the improvements discussed above. As such, the present teachings are not intended to be limiting herein.

The above provides a brief discussion of some potential advantages of the present invention. What follows is an illustrative example of further details of one or more implementations of various embodiments of the present invention. In protein or other biological sample processes, the steps of incubating and washing are very important. To simplify the incubation and washing processes and to integrate those steps into a high throughput fashion, the present inventors have designed a diagnostic sampling platform that uses hydrophobic well structures that can be physically connected to one another by microfluidic channels.

The substrate formats of the present invention can be highly varied. For instance, according to certain embodiments, the protein spots are directly imaged into the wells of the biological compact disc. In yet other embodiments, a conventional well plate can be used in which the protein spots are printed onto an optically flat bottom that has been coated with dielectric layers that provide the quadrature condition. In any event, the substrates are configured to consist of microfluidic systems that deliver the sample to the protein spots in real-time. Useful substrates in accordance with the present teachings include glass substrates (e.g., AR coatings on glass, dielectric stacks on glass, etc.) and silicon substrates (e.g., 120 nm oxide on silicon, 100 nm oxide on silicon, 80 nm oxide on silicon, SiN on silicon, etc.).

As should be understood and appreciated herein, there are many different disc configurations usable with the present invention. For instance, in certain exemplary embodiments, the test pattern on the disc may comprise anywhere from about 10 wells to about 10,000 wells. One specific exemplary disc platform is the Quadraspec Biological Compact Disc system, which includes various disc formats and test patterns (e.g., the 84-well heartworm disc, the 96-well disc, the 108-well disc and the 260-well disc). It should be understood and appreciated that each disc will have different design parameters based on the tests that are being run, e.g., incubation times, well assignments, wash buffers, etc. In other words, the test pattern of the hydrophobic wells on the substrates can vary depending on the desired implementation of the screening procedure to be conducted. As such, various sizes of wells and/or microfluidic channels can be developed for different diagnostic applications in accordance with the present invention.

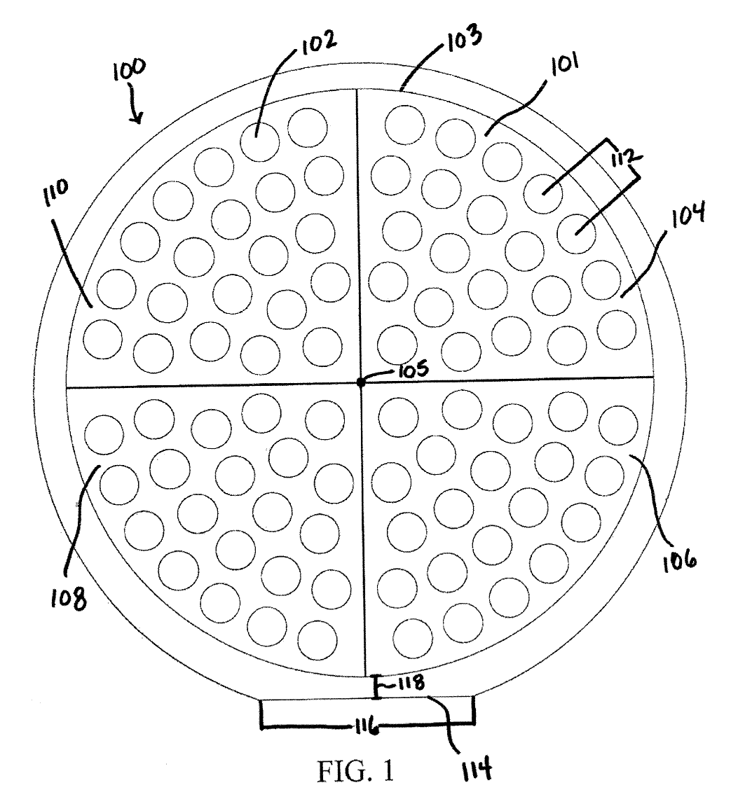

In designing exemplary patterns for the hydrophobic wells discussed herein, various software programs, such as AutoCAD 2006 (Autodesk, Inc., San Rafael, Calif., USA), can be used. For instance, and with reference to FIGS. 1-4, different patterns of hydrophobic wells were designed by using AutoCAD 2006. While the test patterns for the hydrophobic wells of the present invention are described as having various dimensional parameters, it should be understood and appreciated herein that these dimensions have been included for illustrative purposes only and should not be interpreted as limiting the scope of the present teachings. More particularly, those skilled in the relevant art will understand that other such dimensional configurations may be used in accordance with the present teachings without limiting the scope of the hydrophobic well patterns discussed herein.

As explained above, the present invention utilizes a biological compact disc system as the carrier of the diagnostic assays undergoing analysis. The disc, and hence test pattern passes through three processing stages before a diagnostic result is attained. The three processing stages include: 1) disc printing, which includes two steps (i.e., a ‘pad print’ of the disc to do the well boundaries and a ‘protein print,’ which prints the array of antibody spots on the disc; 2) sample processing; and 3) disc reading.

Various definitions used herein to describe the direct printing process of the present invention are as follows: Disc—The carrier of the diagnostic assays and test pattern; Test Pattern—Arrangement of wells on the disc; and Well—Area on the disc for holding diagnostic assays and conducting tests. It should also be understood herein that each printed spot or collection of spots on the disc can serve as an assay. As used herein, “microfluidic channel” refers to a structure adapted to facilitate the precise control of flow of an aqueous system in a desired manner.

As previously noted, processes for manufacturing exemplary discs in accordance with the present invention occur via direct printing methods. One such exemplary process for directly printing the hydrophobic wells involves the use of a Pad Printing Ink Printer machine (XP-13 CE, Pad Print Machinery of VT, Inc., of East Dorset, Vt., USA). According to this process, hydrophobic wells are directly printed onto the disc substrate by printing techniques, such as pad printing techniques or screen printing techniques. In certain exemplary embodiments herein, the hydrophobic wells are printed onto the substrates by pad printing techniques, primarily because these techniques have effective performance standards, particularly in terms of their dimensional pattern specifications and the printing sharpness of the well edges. Moreover, the inks needed to create the desired surface energies and thickness of the wells is much more widely available for such pad printing techniques. While numerous different inks may be used to print the wells in accordance with the present teachings, one such exemplary printing ink is the PLT4G ink available from Pad Print Machinery of VT, Inc. of East Dorset Vt., USA. Other than the pigment itself, 2-methoxy-1-methylethyl and butylglycol acetates (solvents) are the main components of the ink. Since the ink is in itself a mixture, minor changes in composition are unlikely to result in a major change in properties.

A cliche for directly printing the hydrophobic wells onto the disc is made by transferring the AutoCAD file via Adobe Illustrator (Adobe Systems, Inc., San Jose, Calif., USA) to a steel mask. Then, the cliche and ink are loaded to the Pad Printing machine and the pattern printed onto the selected substrate (i.e., either glass or silicon). In general, the cliche is a hard template with a reversed pattern for printing. It is usually manufactured from stainless steel, although it is contemplated as within the scope of the invention that other such durable materials may also be used without straying from the scope of the present invention.

After the patterns are printed on the discs, verification steps can then be implemented to ensure that the wells and microfluidic channels are working correctly. For instance, contact angle measurements (taken using FTA 125 or the FTA 1000 Goniometers, from First Ten Angstroms, Inc., Portsmouth, Va., USA) can be performed on the discs to verify the hydrophobic properties of the print. More particularly, as is known generally within the art, contact angle measurements can be used to indirectly determine surface energies of a disc substrate. In terms of the printing industry, it is of interest to determine whether the ink is creating pearls on the surface or is spreading across the surface. According to the present teachings, it is essential to spread the sample within the well, yet not to allow the sample to spread onto the print. By controlling the contact angles of the print, a sample can be contained within one or more given wells, yet still not bridge any of the barriers between the wells.

During the disc printing process, the disc is held in place while the well test pattern is printed onto the disc. The hydrophobic pattern is printed to create the wells, and a rotational marker is added for the disc reader. Alternatively, a hydrophilic pattern containing a similar rotational marker can be printed on a hydrophobic surface to create hydrophilic well and microfluidic structures. Other required practical elements are then incorporated into the printed pattern. For example, a ‘key’ can be included in the pad print design, which can then be used to determine the disc's orientation. In a later step, the antibodies are added to each of the wells, and the disc can then be further treated and stored before being placed into the sample processor. Once the disc is placed into the sample processor, a pipettor is used to dispense the samples, standards and quenching buffer into the pre-determined wells as desired. Finally, when the disc is advanced to the disc-reading step, the processed disc will be placed into a reader, where the wells are measured for reactions between the sample and the antibodies.

Exemplary illustrations of various disc dimensions and test patterns, which are useful in accordance with the present invention, are now discussed with reference to FIGS. 1-4. In FIG. 1, biological compact disc 100 is illustrated having a test pattern 101 of eighty-four (84) single hydrophobic wells 102. Disc 100 is divided into four equally sized segments or quadrants (see the four quadrants labeled with reference numerals 104, 106, 108, 110), each of which contain twenty-one (21) hydrophobic wells. From the center 105 of the disc's test pattern 101 (i.e., the disc's center of rotation) to its outer edge 103, the hydrophobic wells are arranged in five (5) rows. Each well has a diameter of about 6 mm, while the distance between each well is about 8 mm from center-to-center (see the line labeled with reference numeral 112). The diameter of the disc is about 100 mm±0.5, while the diameter of the test pattern 101 is about 90 mm±0.5. The bottom section of disc 100 has a flat section 114 that is used to hold the disc in place during the processing steps. The length of the flat 114 is about 32.5 mm±2.5 (see the line labeled with reference numeral 116), while the distance between the edge of the flat and the edge of the test pattern 101 is about 3.29 mm (see the line labeled with reference numeral 118). An exemplary thickness of a silicon substrate in accordance with the present invention is about 0.545 mm±0.02, while an exemplary thickness of a glass substrate is about 1.1 mm±0.1.

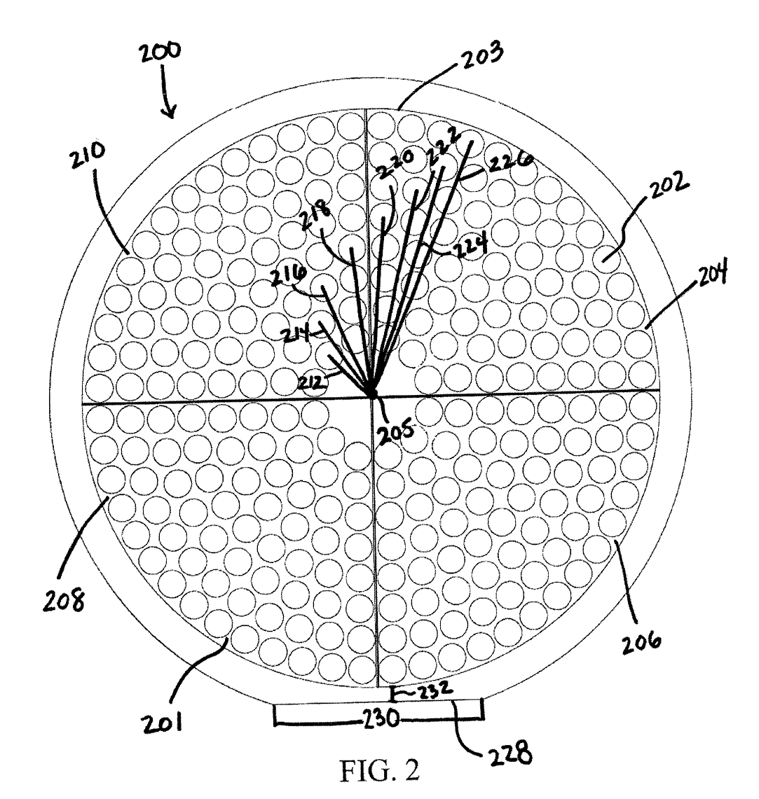

FIG. 2 depicts biological compact disc 200, which has a test pattern 201 of 260 single hydrophobic wells 202. Disc 200 is divided into four equally sized segments or quadrants (see the four quadrants labeled with reference numerals 204, 206, 208, 210), each of which contain sixty-five (65) hydrophobic wells. From the center 205 of the disc's test pattern 201 (i.e., the disc's center of rotation) to its outer edge 203, the hydrophobic wells are arranged in eight (8) rows of concentric circles. The distance from the center of the test pattern 201 to the center of any well located within the first row is about 9.22 mm (see the line labeled with reference numeral 212), while the distance from the center of the test pattern 201 to the center of any well located within the second row is about 13.81 mm (see the line labeled with reference numeral 214). The distance from the center of the test pattern 201 to the center of any well located within the third row is about 18.60 mm (see the line labeled with reference numeral 216), while the distance from the center of the test pattern 201 to the center of any well located within the fourth row is about 23.37 mm (see the line labeled with reference numeral 218). The distance from the center of the test pattern 201 to the center of any well located within the fifth row is about 28.10 mm (see the line labeled with reference numeral 220), while the distance from the center of the test pattern 201 to the center of any well located within the sixth row is about 32.85 mm (see the line labeled with reference numeral 222). Finally, the distance from the center of the test pattern 201 to the center of any well located within the seventh row is about 37.60 mm (see the line labeled with reference numeral 224), while the distance from the center of the test pattern 201 to the center of any well located within the eighth row is about 42.32 mm (see the line labeled with reference numeral 226).

Each well 202 of disc 200 has a diameter of about 4.3 mm. The diameter of the disc is about 100 mm±0.5, while the diameter of the test pattern 201 is about 90 mm±0.5. The flat 228 at the bottom of the disc is about 32.5 mm±2.5 (see the line labeled with reference numeral 230), while the distance between the edge of the flat and the edge of the test pattern is about 2.32 mm (see the line labeled with reference numeral 232). An exemplary thickness of a silicon substrate in accordance with the present invention is about 0.545 mm±0.02, while an exemplary thickness of a glass substrate is about 1.1 mm±0.1.

Various embodiments of the present invention relate to improvements in the capturing and/or enrichment of molecules in a microfluidic channel over/by a capturing surface. Examples of such capturing surfaces include planar surfaces, which can immobilize antibodies. These planar surfaces can be printed by a protein microarray printer. In an assay, a sample is placed over the printed spots and the immunological binding happens through static diffusion of the antigens to the capturing antibodies. Other embodiments of the invention also provide improvements in the functionality of capturing and/or enrichment of molecules in a channel. Examples of various means for achieving this functionality include channel geometries, such as closed capillaries, open channels and channels that contain turbulence or convective elements. By using microfluidic channels, the weakness of the static diffusion of the antigens to the capturing antibodies is overcome by flowing sample over the immobilized antibodies, thus creating more binding events during the assay.

According to certain aspects of the present invention, the limit detection for static incubation/diffusion is about 70 pg/mL. According to this embodiment, the static incubation of a single spot can be determined as follows:

$${c}_{1 / 2} = \frac{{m}_{w}}{π {d}^{2} {L}_{D} {N}_{0} K ( \sqrt{{N}_{s} )}}$$

wherein K=Kinetics of Association (Estimated at 10⁸ for 1 hour incubation for IgG; c_{1/2}=0.8 ng/mL, single spot; ˜115 pg/mL 49 uniform spots; and cₘᵢₙ˜0.5 ng/mL, single spot; ˜70 pg/mL, 49 uniform spots); c=detected concentration; m=molecular weight of antigen; πd²L_{D}=diffusion cylinder volume; N₀=Avagadro's Number; and Nₛ=Number of Ab Spots.

Moreover, according to other exemplary embodiments of the present invention, the limit detection for exemplary microfluidic discs is about <1 pg/mL. According to this exemplary embodiment, for microfluidic incubation of a single spot level of detection a ½ saturation point can be calculated as follows:

$${c}_{1 / 2} = \frac{{m}_{w}}{V ( π {d}^{2} {L}_{D} ) {N}_{0} K ( \sqrt{{N}_{s} )}}$$

wherein V=the number of diffusion volumes passed over spot (c_{1/2}=10 pg/mL, single spot; ˜6 pg/mL 10 uniform spots; and cₘᵢₙ˜4 pg/mL, single spot; ˜1 pg/mL, 10 uniform spots); K=Ab kinetics of association; c=detected concentration; m=molecular weight of antigen; πd²L_{D}=diffusion cylinder volume; N₀=Avagadro's Number; and Nₛ=Number of Ab Spots.

According to one embodiment of the present invention, capillary channels are employed to mobilize a biological sample that contain antigens for binding to specific antibodies on a substrate for detecting bound antibody-antigen pairs. Many different pairs of analytes and binding sites can be employed in a single sample application. The nature of the channels that direct transport of the sample can be dramatically varied in terms of surface chemistry properties, architectural design or the substrates used to form the apparatus. More particularly, according to one aspect of the present invention, a multi-step process for constructing complex Microfluidic Channels to facilitate the advantageous flow control of biological samples is provided. According to this aspect, the multi-step process creates a microfluidic channel that controls the flow of a biological fluid, particularly by altering the surface energy or geometry of the Microfluidic Channels. Moreover, the surface energy of the microfluidic channels are altered by chemically modifying the channel surfaces or by using a second printing material that is different from the hydrophobic inks previously described. Alteration of the microfluidic channel's geometry to control the biological sample flow includes building the height of the channel wall, as well as changing the spacing and/or length of the channels. As geometric and physicochemical properties of Microfluidic Channels contribute to the control of sample flow, it should be understood that as the surface energy of the channel increases, the sample flow rate also increases. Moreover, if a high surface energy channel is employed, it should be understood that by either decreasing the channel spacing or increasing the height of the channel, the rate and energetics of the sample flow will also increase. Conversely, as the surface energy of the channel decreases, the sample flow rate decreases. As such, sample flow rate or energetics of sample flow can be increased by increasing the channel spacing and decreasing the channel wall height. These structural and physicochemical modifications allow for much flexibility in the design of the Microfluidic Channels, particularly with respect to gaining a desired level of sample flow control.

As previously mentioned, one object of the present invention is to construct structures for controlling sample flow or other mass-transfer events. As used herein, sample is defined as an aqueous medium containing one or more analytes. Similarly, other mass transfer events include, but are not limited to, turbulent flow, mixing action, wetting, wicking, dissolving and convective flow. Additionally, some embodiments of the present invention permit precise distribution of the sample to specific substrates as a means of mass transport. This mass transport can occur along preformed hydrophilic channels, otherwise known as Microfluidic Channels, to direct sample transport. As is discussed below, there are generally four features, which define the present microfluidic channels.

First, the physical hydrophilic domains are within structures having defined channel sizes and shapes to thereby direct the flow of the sample. The channels are defined with at least one side or domain that is sufficiently hydrophilic, thereby drawing the sample into the channel for mass transport. The channels also have a hydrophobic domain that defines the limit or boundary of the sample flow. In practice, capillarity occurs with the cooperative effect of the multiple surfaces. An exemplary example of such a structure is a capillary tube in which the liquid contacts a wettable surface at every angle.

From a mass transport standpoint, the present microfluidic channels increase the number of diffusion volumes passing over the capturing surface (e.g., <1 ng/ml demonstrated through static incubation). Moreover, with respect to noise reduction, it has been discovered that the uniform deposition of surface chemistries and antibodies on the surfaces result in good frequency filtering and demodulation capabilities for such high sensitivity assays.

The second defining characteristic of the microfluidic channels is the addition of a hydrophobic structural domain, which is juxtaposed to the hydrophilic domain. This domain defines the boundary area in which the aqueous sample is contained within, i.e., the hydrophobic patterned surface between the channels.

The third defining aspect of the microfluidic channels is the addition of the sample application and receiving reservoirs. In accordance with this aspect of the present invention, the containment of the sample is maintained either by a physical barrier or by a hydrophobic domain that is sufficiently hydrophobic to thereby contain the sample flow. For purposes of the present invention, these design elements can be incorporated together. Moreover, it will be understood by those of ordinary skill in the art that it is possible to construct a microfluidic channel based only on hydrophilic and hydrophobic domains.

The fourth element of the microfluidic channels is the incorporation of macromolecular domains, which are points of immobilized biological or other macromolecules that bind analytes within the sample (e.g., as could be created by a microarray printer). The addition of these molecules to the surface is typically along the hydrophilic domain of the channel. Moreover, these molecules can substantively contribute to the physicochemical properties of the surface. Inherent in the microfluidic system design is the need to avoid interfering with the method of detection of the molecular binding event. This relates to more general considerations of the nature of the microfluidic channels, which will now be discussed in more detail.

In certain exemplary embodiments, the microfluidic channels are more hydrophilic with increasing surface tension of the applied sample. In other words, the surface energy of the hydrophilic domain must be greater than the surface tension of the liquid before capillary action or microfluidic flow is possible. Additionally, for “wettable” surfaces, adjacent structures contribute to capillary action as an inverse function of distance. This is to say that capillary forces increase with decrease in separation. In addition, greater hydrophilic surface area contributes positively to the driving force of capillarity. Wicking of liquids through a wettable substrate, such as fine spun glass fibers, is one exemplary example.

The channel is composed of an inlet and an outlet of defined dimensions. The surface of the channel should be sufficiently hydrophilic or become sufficiently hydrophilic to allow the desired fluid to move by capillarity, sample weight or gravity, surface tension reduction, wetting action, wicking action, chemical modification, other physicochemical modifications or combinations of these forces.

In an analogous manner, it should be noted that containment of the sample can be facilitated by a combination of surface hydrophobicity and the physical structure. As hydrophobicity and/or the surface area that is hydrophobic in nature increases, containment of an aqueous sample is more certain.

Sample flow control is achieved by modification of the physical and chemical nature of the microfluidic channel. Important factors in the design of the microfluidic channel(s) include considering the surface tension of the aqueous liquid that makes up the sample, the physical dimensions of the channel, the surface energy associated with the hydrophilic and hydrophobic domains and other physicochemical features that may dynamically influence the sample flow characteristics. Dynamic microfluidic systems may be employed by introducing surface characteristics, which change on contact with the aqueous sample and is a design element of the present invention.

Other parameters to consider in microfluidic channel design relate to surface modifications. That is to say, various embodiments of the present invention include modifying the substrate surface undergoing a physical or chemical transformation, which thereby introduces convective or turbulent action within the sample. As has been previously discussed, in a completely static assay system that utilizes bimolecular interactions, diffusion can limit the amount of analyte contacting the target-binding site on the substrate. Even small convective fluid movement in the sample will provide a “turnover” to introduce antigen to the substrate surface for binding.

In one aspect of the present invention, surface chemistry is introduced to specific locations on the substrate that will undergo an exothermic reaction on the surface to create a very small, localized heat upon contact with the sample. For example, metal oxidations might be exploited to undergo a reaction once exposed to a sample. It should be understood that it is contemplated as within the scope of the invention to make use of any surface chemistry that includes an exothermic reaction that does not interfere with the assay, and preferably exhibits stability in the dry state and is otherwise stable to the surface chemistry.

In yet another embodiment of the present invention, a chemical transformation can modify the chemical nature of the surface in contact with the fluid containing the sample. The transformation imparts a desired microfluidic action in the sample fluid by the creation of a non-equilibrated state. One specific example observed by one or more of the present inventors was in a system in which the Schiff base hydrolysis could be tracked by the liberation of the hydrophobic compound from the disc surface, which under microscopic observation visibly produced turbulent conditions within the aqueous media. Literally, the sample containing fluid appeared to undergo a dynamic vacillation as the hydrolysis continued. This can be explained by the release of a hydrophobic molecule and immiscible chemical species that is less dense than the water drop in which it is being released.

In yet another embodiment of the present invention, the microfluidic channels are derived from the etching of the surface of the hydrophilic substrate. In this embodiment, a patterning mask to protect the non-etched surface can be employed (but not specifically required). For example, a photoresist can be patterned in a desired configuration and developed to expose the areas to be etched. The etching process can be done by numerous known methods including, but not limited to, chemicals or plasma treatment in the presence of etching gas mixtures. The depth and the shape of the etch can be controlled by the conditions of the specific etching method chosen. If the substrate to be etched is sufficiently hydrophilic in nature or becomes sufficiently hydrophilic because of the process, the channel will facilitate microfluidic or capillary action for a given fluid. If, on the other hand, the etching produces a surface that is not sufficiently hydrophilic to support capillarity, it is possible to treat the surface so-as to impart an acceptably hydrophilicity for microfluidic behavior by means of a second physical or chemical treatment. For example, if the etched surface is silicon or glass in nature, it is possible to treat the substrate surface with silanes or other chemical species, which may be made sufficiently hydrophilic to facilitate microfluidic or capillary action.

Various embodiments of the present invention generally relate to a microfluidic or microfluidic-like apparatus to mobilize biological molecules or analytes to specific target sites for detection. The process of fluid dynamics facilitates transport and subsequent binding and detection of low concentrations of single or multiple analytes contained within a biological sample. In one embodiment, capillary channels are preferably employed to mobilize a biological sample that contains antigens that bind to specific antibodies on a substrate for being detected as a bound antibody-antigen pair. Many different pairs of analytes and binding sites can be employed in a single sample application through the method and apparatus of the present invention. The nature of the channels that direct the transport of the sample can be dramatically varied in terms of surface chemistry properties, architectural design or the substrates used to form the device.

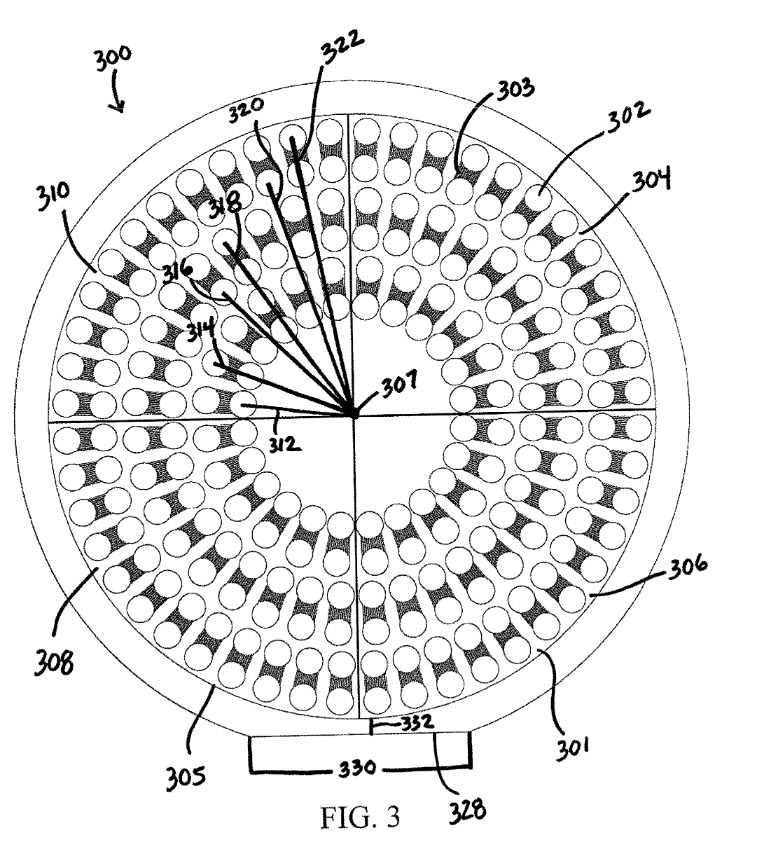

With reference to FIG. 3, biological disc 300 is shown having an exemplary test pattern 301 of 108 double hydrophobic wells. Disc 300 is divided into four equally sized segments or quadrants (see the four quadrants labeled with reference numerals 304, 306, 308, 310), each of which contain twenty-seven (27) double hydrophobic wells. Each well 302 has a diameter of about 4.0 mm and is connected or physically adjoined to another well via a microfluidic channel 303 having a width of approximately 200 μm and a depth of approximately 15 μm. The microfluidic channels contain antibody spots over which the sample is forced to flow by capillary force. This flow creates higher sensitivity of the analytical test by allowing more immunological binding events to happen between antibody and antigen from the sample during the incubation time.

From the center 307 of the disc's test pattern 301 (i.e., the disc's center of rotation) to its outer edge 305, the double hydrophobic wells are arranged in three (3) rows of concentric circles. The distance from the center of the test pattern 301 to the center of any bottom well located within first row is about 16.48 mm (see the line labeled with reference numeral 312), while the distance from the center of the test pattern 301 to the center of any top well located within the first row is about 21.93 mm (see the line labeled with reference numeral 314). The distance from the center of the test pattern 301 to the center of any bottom well located within the second row is about 26.69 mm (see the line labeled with reference numeral 316), while the distance from the center of the test pattern 301 to the center of any top well located within the second row is about 32.14 mm (see the line labeled with reference numeral 318). Finally, the distance from the center of the test pattern 301 to the center of any bottom well located within the third row is about 36.97 mm (see the line labeled with reference numeral 320), while the distance from the center of the test pattern 301 to the center of any top well located within the third row is about 42.41 mm (see the line labeled with reference numeral 322).

The diameter of the disc is about 100 mm±0.5, while the diameter of the test pattern 301 is about 90 mm±0.5. The flat 328 at the bottom of the disc is about 32.5 mm±2.5 (see the line labeled with reference numeral 330), while the distance between the edge of the flat and the edge of the test pattern is about 2.32 mm (see the line labeled with reference numeral 332). An exemplary thickness of a silicon substrate in accordance with the present invention is about 0.545 mm±0.02, while an exemplary thickness of a glass substrate is about 1.1 mm±0.1.

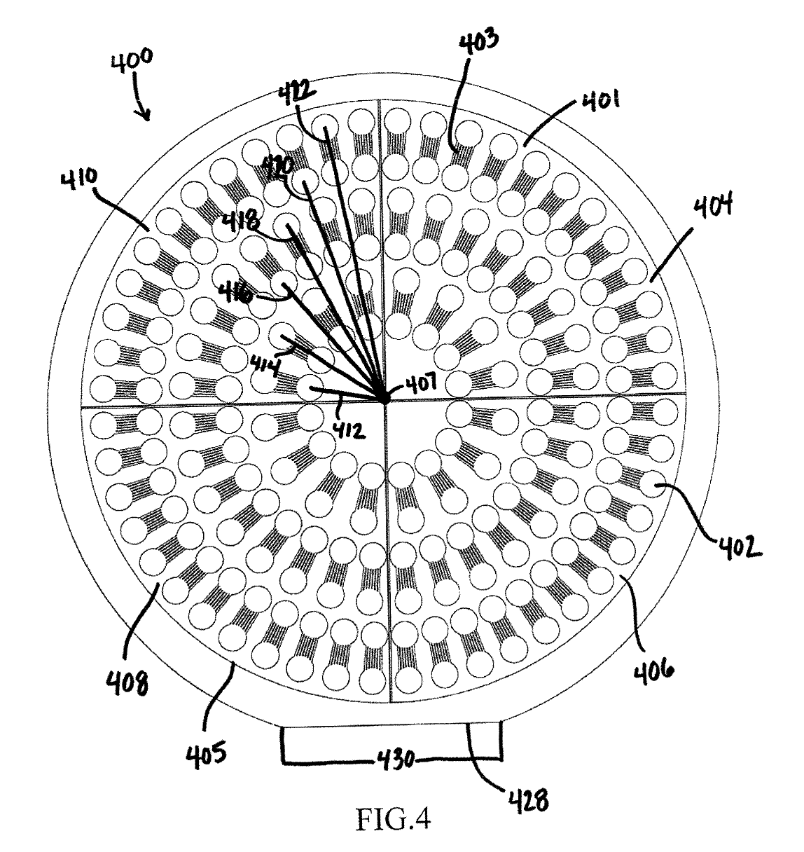

With reference to FIG. 4, biological disc 400 is shown having an exemplary test pattern 401 of 96 double hydrophobic wells. Disc 400 is divided into four equally sized segments or quadrants (see the four quadrants labeled with reference numerals 404, 406, 408, 410), each of which contain twenty-four (24) double hydrophobic wells. Each well 402 has a diameter of about 4.0 mm and is connected or physically adjoined to another well via a microfluidic channel 403 having a width of approximately 250 μm and a depth of approximately 15 μm.

From the center 407 of the disc's test pattern 401 (i.e., the disc's center of rotation) to its outer edge 405, the double hydrophobic wells are arranged in three (3) concentric circles. The distance from the center of the test pattern 401 to the center of any bottom well located within first row is about 11.40 mm (see the line labeled with reference numeral 412), while the distance from the center of the test pattern 401 to the center of any top well located within the first row is about 18.20 mm (see the line labeled with reference numeral 414). The distance from the center of the test pattern 401 to the center of any bottom well located within the second row is about 23.20 mm (see the line labeled with reference numeral 416), while the distance from the center of the test pattern 401 to the center of any top well located within the second row is about 30.00 mm (see the line labeled with reference numeral 418). Finally, the distance from the center of the test pattern 401 to the center of any bottom well located within the third row is about 35.00 mm (see the line labeled with reference numeral 420), while the distance from the center of the test pattern 401 to the center of any top well located within the third row is about 41.8 mm (see the line labeled with reference numeral 422).

The diameter of the disc is about 100 mm±0.5, while the diameter of the test pattern 401 is about 90 mm±0.5. The flat 428 at the bottom of the disc is about 32.5 mm±2.5 (see the line labeled with reference numeral 430), while the distance between the edge of the flat and the edge of the test pattern is about 2.32 mm (see the line labeled with reference numeral 432). An exemplary thickness of a silicon substrate in accordance with the present invention is about 0.545 mm±0.02, while an exemplary thickness of a glass substrate is about 1.1 mm±0.1.

Having now discussed exemplary testing patterns, exemplary methods of manufacturing such testing patterns in accordance with the present invention are provided.

As previously mentioned, the testing patterns used for the substrates of FIGS. 1-4 can be designed for different array applications. The wells can be single or double wells, in addition to being with or without a microfluidic channel connection. This provides the flexibility to make the features suitable for specific applications. While the exemplary embodiments of FIGS. 1-4 can be designed to hold various sample volumes, in certain aspects of the present invention, the sample volumes are in the range of approximately 3-30 μL. It should be understood, however, that these dimensions may be varied and that other sample volumes are contemplated as being within the scope of the invention without straying from the present teachings herein.

In various embodiments of the present invention, a structure having only a single layer can be formed. Embodiments including this feature eliminate sophisticated fabrication steps typically used when constructing microfluidic structures, such as photolithographic techniques for instance. Similarly, the herein disclosed one-step printing process, as, for example, via direct printing by the Pad Printing machine, simplifies the overall manufacturing process and significantly reduces processing time (e.g. such as caused by sophisticated photolithography printing techniques). Roughly, the processing time needed to complete photolithographic techniques may range anywhere from several hours to a couple of days, particularly as these techniques include a series of sophisticated steps (e.g., photomask fabrication steps, photoresist patterning steps, etc.). By comparison, the printing process of the present invention involves a one-step printing technique. Consequently, the overall processing time can be reduced to one or two hours. Additionally, as already noted, the process of the present invention omits several steps when compared to photolithographic methods, such as photomask fabrication and photoresist patterning (including photoresist spinning, alignment and exposure, developing and baking). Moreover, the printing solution of the present invention is also significantly less expensive as compared to photolithography instruments.

The exemplary direct printing patterns of the present invention are monolayer structures, which are created on the surface of the selected substrates. Additionally, all wells and microfluidic channels are typically opened, as opposed to having sealed chambers or tunnels. As such, these embodiments do not require the sophisticated molding and sealing fabrication steps that might otherwise be necessary, thus significantly reducing the processing time.

According to one aspect herein, the direct printing patterns of the present invention involve the fabrication of a multiplicity of layers. According to this exemplary embodiment, a second printed pattern is provided containing the same or different structural features. Additionally, the physicochemical properties of all or portions of the printed structures can be modified by the addition of a second print to create desirable qualities in the containment wells or the microfluidic channels. Microfluidic Channels with tuned surface energies can be created by such second printing techniques with minimal increase of cost or fabrication time.

It should be understood from the above that a wide variety of dimensions and design choices are contemplated as within the scope of the invention. The well size is defined (and designed) to hold not only the sample but also the buffer and washing solutions needed to process the microarray. Since the dot size of the microarray can be smaller than 50 μm, the corresponding well size may vary anywhere from under 2 mm to up to 10 mm or more. Available space within the substrate and the total number of assay wells within the disc determine a well's final dimensions.

Similarly, the number of wells may also vary widely, with a range between 10-1000 likely being possible for a 4 inch (100 mm) diameter wafer. Double wells have a combination of two single wells in one unit connected by microfluidic channels; in this case, the number of wells within the substrate may reach half of the equivalent single well pattern equivalent. The depth of the wells depends on the number of (pad) print hits. Typically, one pass of printing builds up a well that is 6.5 μm deep. A two-hit print produces a well that is 13 μm deep and a three-hit print produces a well 20 μm deep. Embodiments with up to 10 passes, if needed, are contemplated as within the scope of the invention (with pattern degradation being the major concern). It should be understood by those of ordinary skill in the art that the sample volume is determined by the well size. While experimentation data for sample volumes suggest that 3-30 μL may be useful for current patterns as implemented, other sample volumes are contemplated as being within the scope of the invention. Similarly, it should also be understood by those of ordinary skill in the art that when the well size increases or decreases, the sample volume should also be adjusted accordingly.

Other dimensions and features of the present invention may vary widely. For example, the microfluidic channel can span a large range from a few microns to a few hundred microns in width. It should be understood that a single microfluidic channel may connect double (or multiple) well embodiments of the present invention. That is to say, embodiments with a single microfluidic channel instead of multiple microfluidic channels are contemplated as being within the scope of the invention. The embodiments with multiple channels are useful for accommodating additional analytes in multiplexed scenarios.

While an exemplary embodiment incorporating the principles of the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A diagnostic platform, comprising:
a substrate having a plurality of hydrophobic wells formed thereon by directly printing a test pattern onto the substrate, each of the hydrophobic wells being adapted to contain a biological sample for conducting a diagnostic assay to identify analytes within the biological sample;
wherein the test pattern is divided into equally sized quadrants, each containing an equal number of hydrophobic wells.

2. The diagnostic platform of claim 1, wherein the test pattern comprises between about 10 and about 10,000 single hydrophobic wells.

3. The diagnostic platform of claim 2, wherein the test pattern comprises 84 single hydrophobic wells, each well having a diameter of about 6 mm.

4. The diagnostic platform of claim 2, wherein the test pattern comprises 260 single hydrophobic wells, each having a diameter of about 4.3 mm.

5. The diagnostic platform of claim 1, wherein the test pattern comprises between about 10 and about 10,000 double hydrophobic wells, each pair of double wells being connected by at least one microfluidic channel.

6. The diagnostic platform of claim 5, wherein the test pattern comprises 108 double hydrophobic wells, each well having a diameter of about 4 mm.

7. The diagnostic platform of claim 6, wherein the at least one microfluidic channel has a width of about 200 μm and a depth of about 15 μm.

8. The diagnostic platform of claim 5, wherein the test pattern comprises 96 double hydrophobic wells, each well having a diameter of about 4 mm.

9. The diagnostic platform of claim 8, wherein the at least one microfluidic channel has a width of about 250 μm and a depth of about 15 μm.

10. The diagnostic platform of claim 5, wherein the at least one microfluidic channel is adapted to facilitate the transport of the biological sample to the pair of double hydrophobic wells for enabling a biomolecular binding interaction.

11. The diagnostic platform of claim 10, wherein the biomolecular binding interaction comprises at least one of an antibody-antigen interaction, a DNA hybridization interaction, a RNA hybridization interaction, a DNA-protein interaction, an RNA-protein interaction, a cell surface constituent interaction, an enzymatic interaction, a substrate interaction and a macromolecule interaction.

12. The diagnostic platform of claim 5, wherein the at least one microfluidic channel further comprises a planar capturing surface configured to immobilize antibodies printed thereon.

13. The diagnostic platform of claim 12, wherein the printed antibodies are adapted to cause immunological binding of antigens within the biological sample to the printed antibodies through static diffusion of the antigens to the antibodies.

14. The diagnostic platform of claim 5, wherein the at least one microfluidic channel further comprises a capturing surface selected from at least one of closed capillaries, open channels and channels containing turbulence or convective elements, the capturing surface being configured to immobilize antibodies printed thereon.

15. The diagnostic platform of claim 14, wherein the printed antibodies are adapted to cause immunological binding of antigens within the biological sample to the printed antibodies by overflowing the sample onto the capturing surface.

16. The diagnostic platform of claim 1, wherein the substrate is a biological compact disc formed of at least one of silicon and glass and has a diameter of about 100 mm.

17. The diagnostic platform of claim 1, wherein the test pattern is directly printed onto the substrate by a one-step printing technique selected from at least one of a pad printing technique and a screen printing technique.

18. The diagnostic platform of claim 17, wherein the one-step printing technique comprises directly printing the hydrophobic wells onto the substrate with an ink including 2-methoxy-1-methylethyl and butylgycol acetates.

19. A diagnostic platform for identifying analytes in a biological sample, comprising:
a substrate having a test pattern of between about 10 and about 10,000 hydrophobic wells, the test pattern being directly printed onto the substrate; and
a microfluidic channel adapted to connect a pair of hydrophobic wells within the test pattern, the microfluidic channel being adapted to facilitate the transport of the biological sample to the pair of hydrophobic wells for enabling a biomolecular binding interaction.

20. The diagnostic platform of claim 19, wherein the test pattern comprises 108 double hydrophobic wells, each well having a diameter of about 4 mm.

21. The diagnostic platform of claim 20, wherein the microfluidic channel has a width of about 200 μm and a depth of about 15 μm.

22. The diagnostic platform of claim 19, wherein the test pattern comprises 96 double hydrophobic wells, each well having a diameter of about 4 mm.

23. The diagnostic platform of claim 22, wherein the microfluidic channel has a width of about 250 μm and a depth of about 15 μm.

24. The diagnostic platform of claim 19, wherein the substrate is a biological compact disc formed of at least one of silicon and glass and has a diameter of about 100 mm.

25. The diagnostic platform of claim 19, wherein the direct printing of the test pattern onto the substrate comprises a one-step printing technique selected from at least one of a pad printing technique and a screen printing technique.

26. The diagnostic platform of claim 25, wherein the one-step printing technique comprises directly printing the hydrophobic wells onto the substrate with an ink including 2-methoxy-1-methylethyl and butylgycol acetates.

27. The diagnostic platform of claim 19, wherein the biomolecular binding interaction comprises at least one of an antibody-antigen interaction, a DNA hybridization interaction, a RNA hybridization interaction, a DNA-protein interaction, an RNA-protein interaction, a cell surface constituent interaction, an enzymatic interaction, a substrate interaction and a macromolecule interaction.

28. The diagnostic platform of claim 19, wherein the test pattern is divided into equally sized quadrants, each containing an equal number of hydrophobic wells.

29. The diagnostic platform of claim 19, wherein the at least one microfluidic channel further comprises a planar capturing surface configured to immobilize antibodies printed thereon.

30. The diagnostic platform of claim 29, wherein the printed antibodies are adapted to cause immunological binding of antigens within the biological sample to the printed antibodies through static diffusion of the antigens to the antibodies.

31. The diagnostic platform of claim 19, wherein the at least one microfluidic channel further comprises a capturing surface selected from at least one of closed capillaries, open channels and channels containing turbulence or convective elements, the capturing surface being configured to immobilize antibodies printed thereon.

32. The diagnostic platform of claim 31, wherein the printed antibodies are adapted to cause immunological binding of antigens within the biological sample to the printed antibodies by overflowing the sample onto the capturing surface.

33. A process for preparing a diagnostic platform for identifying analytes in a biological sample, comprising:
directly printing a test pattern of hydrophobic wells onto a substrate, the test pattern being printed onto the substrate by a one-step printing technique selected from at least one of pad printing and screen printing;
imaging protein spots into the hydrophobic wells, the protein spots being configured to conduct a diagnostic assay on the biological sample within the wells to identify analytes contained therein; and
delivering the sample to the protein spots via at least one microfluidic channel coupled to a pair of hydrophobic wells printed on the substrate.

34. The process of claim 33, wherein directly printing a test pattern of hydrophobic wells onto the substrate comprises printing a test pattern of 108 double hydrophobic wells, each well having a diameter of about 4 mm.

35. The process of claim 34, wherein the at least one microfluidic channel has a width of about 200 μm and a depth of about 15 μm.

36. The process of claim 33, wherein directly printing a test pattern of hydrophobic wells onto the substrate comprises printing a test pattern of 96 double hydrophobic wells, each well having a diameter of about 4 mm.

37. The process of claim 36, wherein the at least one microfluidic channel has a width of about 250 μm and a depth of about 15 μm.

38. The process of claim 33, wherein the substrate is a biological compact disc formed of at least one of silicon and glass and has a diameter of about 100 mm.

39. The process of claim 33, wherein the one-step printing technique comprises directly printing the hydrophobic wells onto the substrate with an ink including 2-methoxy-1-methylethyl and butylgycol acetates.

40. The process of claim 43, wherein the test pattern is divided into equally sized quadrants, each containing an equal number of hydrophobic wells.

41. The diagnostic platform of claim 33, wherein the at least one microfluidic channel further comprises a planar capturing surface configured to immobilize antibodies printed thereon.

42. The diagnostic platform of claim 41, wherein the printed antibodies are adapted to cause immunological binding of antigens within the biological sample to the printed antibodies through static diffusion of the antigens to the antibodies.

43. The diagnostic platform of claim 42, wherein the at least one microfluidic channel further comprises a capturing surface selected from at least one of closed capillaries, open channels and channels containing turbulence or convective elements, the capturing surface being configured to immobilize antibodies printed thereon.

44. The diagnostic platform of claim 43, wherein the printed antibodies are adapted to cause immunological binding of antigens within the biological sample to the printed antibodies by overflowing the sample onto the capturing surface.
